(19) United States
(12) Patent Application Publication
(10) Pub. No.: US 2018/0187121 A1
(43) Pub. Date: Jul. 5, 2018

(54) METHOD FOR PRODUCING ONE OR MORE OIL-SOLUBLE BIOPRODUCTS

(71) Applicant: White Dog Labs, Inc., New Castle, DE (US)

(72) Inventors: Aharon M. EYAL, Jerusalem (IL); Bryan P. TRACY, Wilmington, DE (US); Christopher Joseph MCWILLIAMS, Somerville, MA (US)

(73) Assignee: White Dog Labs, Inc., New Castle, DE (US)

(21) Appl. No.: 15/736,581

(22) PCT Filed: Jun. 14, 2016
(86) PCT No.: PCT/US16/37385

§ 371 Date: Dec. 14, 2017

Related U.S. Application Data
(60) Provisional application No. 62/175,687, filed on Jun. 15, 2015.

(51) Int. Cl. C11B 1/10; C07C 7/10; C12P 7/64; C12P 5/02

(52) U.S. Cl. CPC C11B 1/104; C07C 7/10; C07C 11/18; C12P 5/026; C12P 7/6409

(57) ABSTRACT

Disclosed are methods for making one or more bioproducts such as isoprene derivatives, fatty acids, fatty acid derivatives, fatty alcohols, fatty alcohol derivatives, mixtures thereof, and compositions having one or more such bioproducts with microorganisms and for separating the bioproduct(s) or bioproduct composition(s). The methods include providing an extractant having a solvent with a boiling point at atmospheric pressure of less than 20° C.; feeding an aqueous suspension of cells that have produced the bioproduct(s) into a pressurized column; feeding the extractant into the pressurized column; removing an extract having the extractant and the bioproduct(s) from said pressurized column; and separating the extractant from the removed extract to obtain a separated oil-soluble composition having the bioproduct.

### CROSS-REFERENCE TO RELATED APPLICATIONS

This application claims priority to U.S. Provisional Application No. 62/175,687, filed Jun. 15, 2015, which application is incorporated by reference herein in its entirety.

### FIELD OF INVENTION

The invention relates to the field of production of bioproducts such as isoprene derivatives, fatty acids, fatty acid derivatives, fatty alcohols, fatty alcohol derivatives, glycolipids, mixtures thereof, and compositions comprising such bioproducts with a microorganism, including bacteria, fungi and algae.

### BACKGROUND

Oil-soluble bioproducts such as isoprene derivatives, fatty acids, fatty acid derivatives, fatty alcohols, fatty alcohol derivatives and glycolipids are in high demand in several industry sectors, both as target compound precursors and as final products. However, conventional methods for producing and separating oil-soluble bioproducts are inefficient and/or expensive. In many cases, at least a fraction of the bioproduct is intracellular and its high-yield recovery is mass-transfer limited, even in cases where the cells are ruptured. Recovery from separated cell mass adds to the costs of handling solid matter. Accordingly, there is a need for efficient and less expensive methods for producing such oil-soluble bioproducts and for separating them from the medium in which they are produced.

### SUMMARY OF THE INVENTION

Provided herein is a method for producing an oil-soluble bioproduct composition, comprising (i) culturing a host microorganism genetically modified for increased production of an oil-soluble bioproduct to form an aqueous suspension of cells comprising said bioproduct, which bioproduct is at least 30% intracellular; (ii) providing an extractant comprising a solvent, which solvent has a boiling point at atmospheric pressure of less than 20° C. (degrees Celsius); (iii) feeding said aqueous suspension into a pressurized column through a first input port; (iv) feeding said extractant into said pressurized column through a second input port; (v) removing an extract from said pressurized column through a first output port, wherein said removed extract comprises at least a fraction of said extractant and at least of fraction of said bioproduct; (vi) removing a raffinate from said pressurized column through a second output port, wherein said removed raffinate is bioproduct-depleted; and (vii) separating extractant from said removed extract to obtain a separated oil-soluble composition comprising the bioproduct and a separated extractant; wherein a. the concentration of cells in the aqueous suspension fed to the pressurized column is in the range between 0.1 wt % (weight percent) and 10 wt % based on dry-cell weight; b. the concentration of said bioproduct in said aqueous suspension fed to the pressurized column is in the range between 0.01 wt % and 1 wt %; c. the solubility of said bioproduct in water at ambient pressure and 20° C. is less than 2 wt %; d. the solvent forms at least 70 wt % of said provided extractant; e. the solubility of said solvent in water at ambient pressure and 20° C. is less than 2 wt %; f. said removed extract comprises water at less than 20 wt %; and g. said removed extract is free of cells or comprises cells at a dry-cell weight that is less than 10 wt % of the dry-cell weight of the aqueous suspension.

Also provided is such a method, wherein said bioproduct is an isoprene derivative, fatty acid, fatty acid derivative, fatty alcohol, fatty alcohol derivative, glycolipid, and/or mixtures thereof.

Also provided is such a method, wherein said microorganism is selected from the group consisting of bacteria, yeast, and microalgae.

Also provided is such a method, wherein said culturing comprises fermentation, wherein said fermentation forms a fermentation broth and wherein said aqueous suspension comprises said fermentation broth.

Also provided is such a method that further comprises rupturing cells in said aqueous suspension prior to feeding the aqueous suspension into said pressurized column.

In an embodiment, the method may further comprise adjusting the cell concentration in said aqueous suspension prior to feeding the aqueous suspension into said pressurized column, which adjusting may comprise at least one of water addition and water removal.

Also provided is such a method, wherein said solvent is selected from CO₂, butene, butane, and/or propane.

Also provided is such a method, wherein the temperature of said column, the pressure of said column or both are within the super-critical range of said solvent.

Also provided is such a method, wherein said extractant and said aqueous suspension are fed at aqueous suspension/extractant weight/weight ratio in the range between 1/10 and 10/1.

Also provided is such a method, wherein said aqueous suspension is fed at a flow rate in the range between 100 L/h and 10,000 L/hr (Liters per hour).

Also provided is such a method, wherein at least 80% of the bioproduct present in said aqueous suspension after culturing is recovered in the separated oil-soluble composition.

In an embodiment, the method may further comprise purifying said bioproduct by separating co-extracted co-product lipids or a fraction thereof from the separated oil-soluble composition. In an embodiment, such a method is provided, wherein said purifying comprises extraction with a selective extractant. In an embodiment, said selective extractant may comprise said solvent. In an embodiment, such a method is provided, wherein said selective extractant comprises CO₂.

Also provided is a method as described above, wherein providing an extractant comprises providing a recycled extractant previously utilized in a method for producing an oil-soluble bioproduct or an oil-soluble bioproduct composition.

Also provided is a method as described above, wherein said first output port is above said first input port. Also provided is a method as described above, wherein said second output port is under said second input port.

Also provided is a method for producing an oil-soluble bioproduct, comprising: (i) culturing a host microorganism genetically modified for increased production of an oil-soluble bioproduct to form an aqueous suspension of cells comprising said bioproduct, which bioproduct is at least 30% intracellular; (ii) providing an extractant comprising a solvent, which solvent has a boiling point at atmospheric pressure of less than 20° C.; (iii) feeding said aqueous suspension into a pressurized column through a first input port; (iv) feeding said extractant into said pressurized column through a second input port; (v) removing an extract from said pressurized column through a first output port, wherein said removed extract comprises at least a fraction of said extractant and at least a fraction of said bioproduct; (vi) removing a raffinate from said pressurized column through a second output port, wherein said removed raffinate is bioproduct-depleted; (vii) separating extractant from said removed extract to obtain a separated oil-soluble composition comprising the bioproduct and a separated extractant; and (viii) obtaining an oil-soluble bioproduct from the separated oil-soluble composition; wherein (a) the concentration of cells in the aqueous suspension fed to the pressurized column is in the range between 0.1 wt % and 10 wt % based on dry-cell weight; (b) the concentration of said bioproduct in said aqueous suspension fed to the pressurized column is in the range between 0.01 wt % and 1 wt %; (c) the solubility of said bioproduct in water at ambient pressure and 20° C. is less than 2 wt %; (d) the solvent forms at least 70 wt % of said provided extractant; (e) the solubility of said solvent in water at ambient pressure and 20° C. is less than 2 wt %; (f) said removed extract comprises water at less than 20 wt %; and (g) said removed extract is free of cells or comprises cells at a dry-cell weight that is less than 10 wt % of the dry-cell weight of the aqueous suspension.

### BRIEF DESCRIPTION OF THE DRAWINGS

FIG. 1 shows an embodiment of the pressurized column for extraction. One possible configuration of the first and second input ports and of the first and second output ports is provided.

FIG. 2 shows a flow diagram for one possible method for producing and separating an oil-soluble bioproduct.

### DETAILED DESCRIPTION

The methods described herein provide time-saving and efficient processes for making and separating one or more oil-soluble bioproducts or bioproduct compositions. Superior efficiencies and time savings may be obtained by avoiding a separate cell harvesting step and the high costs associated with such a step. A separate cell harvesting step may be omitted because extraction of the one or more oil-soluble bioproducts or bioproduct compositions may be performed while the cells are in suspension. This avoids not only the effort involved in harvesting the cells prior to extraction, but also expensive equipment necessary for collection of a solid cell mass. Instead, processes as described herein may take advantage of the ability to carry cells over in an aqueous phase to provide a continuous approach that allows production and separation cycle time to be drastically decreased.

As used herein, the singular forms “a,” “an,” and “the” include the plural reference unless the context clearly dictates otherwise. For example, reference to “a bioproduct” would also mean that mixtures of one or more bioproducts can be present unless specifically excluded.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

Unless otherwise stated, a reference to a compound or component includes the compound or component by itself, as well as in combination with other compounds or components, such as mixtures of compounds.

Unless otherwise indicated, all percentages are weight percent.

The various embodiments disclosed herein can be used separately and in various combinations unless specifically stated to the contrary.

Provided is a method for producing an oil-soluble bioproduct composition, comprising (i) culturing a host microorganism genetically modified for increased production of an oil-soluble bioproduct to form an aqueous suspension of cells comprising said bioproduct, which bioproduct is at least 30% intracellular; (ii) providing an extractant comprising a solvent, which solvent has a boiling point at atmospheric pressure of less than 20° C.; (iii) feeding said aqueous suspension into a pressurized column through a first input port; (iv) feeding said extractant into said pressurized column through a second input port; (v) removing an extract from said pressurized column through a first output port, wherein said removed extract comprises at least a fraction of said extractant and at least a fraction of said bioproduct; (vi) removing a raffinate from said pressurized column through a second output port, wherein said removed raffinate is bioproduct-depleted; and (vii) separating extractant from said removed extract to form a separated oil-soluble composition and a separated extractant; wherein a. the concentration of cells in the aqueous suspension fed to the pressurized column may be in the range between 0.1 wt % and 10 wt % based on dry-cell weight; b. the concentration of said bioproduct in said aqueous suspension fed to the pressurized column may be in the range between 0.01 wt % and 1 wt %; c. the solubility of said bioproduct in water at ambient pressure and 20° C. may be less than 2 wt %; d. the solvent may form at least 70 wt % of said provided extractant; e. the solubility of said solvent in water at ambient pressure and 20° C. may be less than 2 wt %; f. said removed extract may comprise water at less than 20 wt %; and g. said removed extract may be free of cells or comprise cells at a dry-cell weight that is less than 5 wt % of the dry-cell weight of aqueous suspension.

Also provided is a method for producing an oil-soluble bioproduct, comprising: (i) culturing a host microorganism genetically modified for increased production of an oil-soluble bioproduct to form an aqueous suspension of cells comprising said bioproduct, which bioproduct is at least 30% intracellular; (ii) providing an extractant comprising a solvent, which solvent has a boiling point at atmospheric pressure of less than 20° C.; (iii) feeding said aqueous suspension into a pressurized column through a first input port; (iv) feeding said extractant into said pressurized column through a second input port; (v) removing an extract from said pressurized column through a first output port, wherein said removed extract comprises at least a fraction of said extractant and at least a fraction of said bioproduct; (vi) removing a raffinate from said pressurized column through a second output port, wherein said removed raffinate is bioproduct-depleted; (vii) separating extractant from said removed extract to obtain a separated oil-soluble composition comprising the bioproduct and a separated extractant; and (viii) obtaining an oil-soluble bioproduct from the separated oil-soluble composition; wherein (a) the concentration of cells in the aqueous suspension fed to the pressurized column may be in the range between 0.1 wt % and 10 wt % based on dry-cell weight; (b) the concentration of said bioproduct in said aqueous suspension fed to the pressurized column may be in the range between 0.01 wt % and 1 wt %; (c) the solubility of said bioproduct in water at ambient pressure and 20° C. may be less than 2 wt %; (d) the solvent may form at least 70 wt % of said provided extractant; (e) the solubility of said solvent in water at ambient pressure and 20° C. may be less than 2 wt %; (1) said removed extract may comprise water at less than 20 wt %; and (g) said removed extract may be free of cells or may comprise cells at a dry-cell weight that is less than 10 wt % of the dry-cell weight of the aqueous suspension.

According to an embodiment, said bioproduct may be comprise an isoprene derivative, a fatty acid, a fatty acid derivative, a fatty alcohol, a fatty alcohol derivative, a glycolipid, and/or combinations or mixtures thereof. According to an embodiment, said isoprene derivative may comprise a carotenoid, a colorant, an antioxidant, a flavor, a fragrance, a farnesene and/or a terpenoid. According to an embodiment, said carotenoid may comprise beta-carotene, zeaxanthin, lutein, astaxanthin and lycopene. According to an embodiment, said fatty acid may comprise saturated and unsaturated fatty acids, mono-carboxylic fatty acids and di-carboxylic fatty acids, unsaturated with a single double bond, two double bonds, or multiple double bonds located in various locations along the molecule. According to an embodiment, said fatty acid may comprise an omega-3 fatty acid. According to an embodiment, said fatty acid may comprise a polyunsaturated mono-carboxylic fatty acid. According to an embodiment, said fatty acid may comprise a polyunsaturated di-carboxylic fatty acid. According to an embodiment, said fatty acid derivative may comprise a fatty acid ester, e.g. methyl or ethyl esters. According to an embodiment, said fatty acid derivative may comprise a glycolipid. According to an embodiment, said glycolipid may comprise a rhamnolipid. According to an embodiment, said bioproduct is astaxanthin. According to an embodiment, the solubility of said bioproduct in water at ambient pressure and at 20° C. is less than 1.5 wt %; less than 1.2 wt %, less than 0.9 wt %; less than 0.7 wt %, less than 0.5 wt %, less than 0.4 wt %; less than 0.3 wt %, less than 0.2 wt % or less than 0.1 wt %.

According to an embodiment, said host microorganism is selected from the group consisting of bacteria, yeast and microalgae. According to an embodiment, said microorganism is genetically modified. For example, the organism may be genetically modified to increase or decrease expression of an enzyme or substrate involved in the metabolism or generation of a target bioproduct.

According to an embodiment, said microorganism is genetically modified Escherichia coli. According to an embodiment, said bioproduct is astaxanthin and said microorganism is genetically modified E. coli. Accordingly to an embodiment, said microorganism is a genetically modified yeast, such as genetically modified Saccharomyces cerevisiae, a Saccharomycopsis spp., a Pichia spp., or a Schwanniomyces spp. According to an embodiment, said microorganism is a genetically modified microalgae, such as a microalgae belonging to the Chlorella or volvox genuses, C. reinhardtii, or the genus Scenedesmus.

According to an embodiment, said culturing comprises fermentation, said fermentation forms a fermentation broth and said aqueous suspension comprises said fermentation broth. According to an embodiment, said fermentation utilizes a carbon source and a nitrogen source and said broth comprises at least one of a residual carbon source and a residual nitrogen source.

According to an embodiment, said bioproduct is at least 30% intracellular, at least 40% intracellular, at least 50%, at least 60%, at least 70%, at least 80%, at least 90% or at least 95%. Viewed another way, according to an embodiment, at least 30% of the total amount of bioproduct produced by the method remains inside the host microorganism and the remaining not more than 70% of produced bioproduct is present outside the host cells in the fermentation broth of the aqueous suspension.

According to an embodiment, the concentration of cells in the aqueous suspension fed to the pressurized column may be greater than 0.1 wt %, greater than 0.5 wt %, greater than 1 wt %, greater than 2 wt %, greater than 3 wt %, or greater than 4 wt %, based on dry-cell weight. According to an embodiment, the concentration of cells in the aqueous suspension fed to the pressurized column may be less than 9.5 wt %, less than 9 wt %, less than 8 wt %, less than 7 wt %, less than 6 wt % or less than 5 wt % based on dry-cell weight.

According to an embodiment, the method may further comprise adjusting the cell concentration in said aqueous suspension. The cell concentration may be adjusted prior to feeding the aqueous suspension into the pressurized column. The cell concentration in the aqueous suspension may be adjusted to the range between 0.1 wt % and 10 wt % based on the dry-cell weight of the cells.

According to an embodiment, the concentration of said bioproduct in the aqueous suspension fed to the pressurized column may be greater than 0.01 wt %, greater than 0.03 wt %, greater than 0.05 wt %, greater than 0.08% greater than 0.1 wt %, or greater than 0.2 wt %. According to an embodiment, the concentration of said bioproduct in the aqueous suspension fed to the pressurized column may be less than 0.95 wt %, less than 0.9 wt %, less than 0.8 wt %, less than 0.7 wt %, less than 0.6 wt %, or less than 0.5 wt %. As used herein the concentration of said bioproduct in the aqueous suspension refers to the combined amounts of intracellular and extracellular bioproduct.

The method of the present invention may further comprise providing an extractant comprising a solvent having a boiling point at atmospheric pressure of less than 20° C. According to an embodiment, the boiling point at atmospheric pressure may be less than 15° C., less than 10° C., less than 5° C., or less than 0° C. The solubility of said solvent in water at ambient pressure and at 20° C. may be less than 2 wt %. According to an embodiment, the solubility of said solvent in water at ambient pressure and 20° C. may be less than 1.5 wt %, less than 1.2 wt %, less than 0.9 wt %, less than 0.7 wt %, less than 0.5 wt %, less than 0.4 wt %, less than 0.3 wt %, less than 0.2 wt %, or less than 0.1 wt %. According to an embodiment, the solubility of said solvent in water at ambient pressure and 20° C. is at least 100 ppm, is at least 500 ppm, is at least 1000 ppm, is at least 1500 ppm, or is at least 2000 ppm. In an embodiment, said solvent may be water-insoluble.

The solvent may form at least 70 wt % of said extractant. According to an embodiment, it forms at least 80 wt % of said extractant, at least 85 wt %, at least 90 wt %, or at least 95 wt %. According to an embodiment, said solvent is recycled. According to an embodiment, providing an extractant may comprise providing a recycled extractant previously utilized in a method for producing an oil-soluble product.

According to an embodiment, said solvent may be CO₂, butene, butane, propane or propene. According to an embodiment, said butene may be at least one of 1-butene, 2-butene, and iso-butene. According to an embodiment, said solvent may comprise a mixture of butene, an ether, and optionally a third component. According to an embodiment, said ether is dimethyl ether. According to an embodiment, said solvent comprises a mixture of CO₂, a water-soluble solvent and optionally a third component. According to an embodiment, the solubility of said water-soluble solvent in water at ambient pressure and 20° C. is at least 30 wt %. According to an embodiment, said water-soluble solvent is ethanol.

The method of the present invention may further comprise feeding said aqueous suspension into a pressurized column through a first input port and feeding said extractant into said pressurized column through a second input port, which second input port is under said first input port. According to an embodiment, the fed aqueous suspension flows downwards, the fed extractant flows upwards and the two flows are counter-current.

According to an embodiment, the method may further comprise adjusting the cell concentration in said aqueous suspension prior to feeding the aqueous suspension into said pressurized column, which adjusting comprises at least one of water addition and water removal.

According to an embodiment, the method may further comprise rupturing cells in said aqueous suspension prior to feeding the aqueous suspension into said pressurized column. Options for rupturing cells include milling or sonication or exposure to rupturing agents.

According to an embodiment, above a certain temperature and pressure, the solvent may be in a super-critical form. According to an embodiment, the temperature of said column is above that temperature, i.e. within the super-critical range of said solvent. According to an embodiment, the pressure of said column is above that pressure, i.e. within the super-critical range of said solvent. According to an embodiment, both the temperature and the pressure of said column are within the super-critical range of said solvent.

According to an embodiment, said aqueous suspension may be fed at a flow rate in the range between 100 L/h (liter per hour) and 10,000 L/hr. According to an embodiment, said aqueous suspension is fed at a linear flux of in the range between 0.5-80 m/h (meter per hour).

According to an embodiment, said extractant and said aqueous suspension may be fed at aqueous suspension/extractant weight/weight ratio in the range between 1/10 and 10/1. According to an embodiment, said weight/weight ratio is at least 3/10, at least 1/2, at least 3/4, at least 1, at least 2, at least 3, at least 4, or at least 5. According to an embodiment, said weight/weight ratio is less than 8, less than 6, less than 5, less than 4, less than 3, less than 2, or less than 1.

In an embodiment, the method provided may further comprise removing an extract from said pressurized column through a first output port, wherein said removed extract comprises said extractant and said bioproduct and less than 20 wt % water. According to an embodiment, the water content of said extract is less than 10 wt %, less than 8 wt %, less than 6 wt %, less than 4 wt %, less than 2 wt %, less than 1 wt %, or less than 0.5 wt %. Said removed extract may be free of cells or comprise cells at a dry-cell weight that is less than 5 wt % of the dry-cell weight of the aqueous suspension, less than 4 wt %, less than 2 wt %, less than 1 wt %, less than 0.5 wt %, less than 0.2 wt %, less than 0.1 wt % or less than 0.05 wt %.

In an embodiment, the method provided may further comprise removing a raffinate from said pressurized column through a second output port, wherein said removed raffinate is bioproduct-depleted. According to the method, said bioproduct may be extracted, i.e. transferred from said aqueous suspension to said extractant to form said extract and said bioproduct-depleted raffinate. According to an embodiment, at least 50% of the bioproduct may be extracted, at least 60%, at least 70%, at least 80%, at least 90%, at least 95%, at least 97%, at least 98% or at least 99%.

According to an embodiment, at least a fraction of the raffinate may be reused in said culturing. According to an embodiment said culturing is fermentation and is conducted in a fermentor fed by a carbon source and a nitrogen source. According to an embodiment, at least a fraction of the raffinate may be used for dissolving or diluting said carbon source.

According to an embodiment, said method may be performed with an extraction column. The extraction column may be pressurized. In an embodiment, the extraction column may be a high pressure column. The extraction column may have two, three or more input ports. The extraction column may also or alternatively have two, three or more output ports. According to an embodiment, said first output port may be configured above said first input port. According to another embodiment, said second output port may be configured below said second input port. According to an embodiment, a first input port and a second input port may be arranged below a first output port and above a second output port.

In an embodiment, the method provided may further comprise separating extractant from said removed extract to obtain a separated oil-soluble composition and a separated extractant. According to an embodiment, separating extractant may comprise pressure reduction, temperature elevation or both. According to an embodiment, pressure is reduced to atmospheric pressure. According to an embodiment, providing an extractant may comprise providing said separated extractant.

According to an embodiment, at least 80% of the bioproduct present in said aqueous suspension, i.e., the combined amount of bioproduct present both intracellularly and extracellularly in the aqueous suspension after culturing, may be recovered in the separated oil-soluble composition, at least 85%, at least 90%, at least 95%, at least 97%%, at least 98%, or at least 99%.

According to an embodiment, said aqueous suspension may comprise co-product lipids. According to an embodiment, at least a fraction of said co-product lipids are intracellular. According to an embodiment, at least a fraction of said co-product lipids are co-extracted with said oil-soluble bioproduct. According to an embodiment, at least a fraction of said co-extracted co-product lipids may be present in said oil-soluble bioproduct composition. According to an embodiment, said oil-soluble composition may comprise a mixture of said oil-soluble bioproduct and co-extracted co-product lipids. According to an embodiment, said oil-soluble composition may comprise a mixture of astaxanthin and co-extracted cell lipids. According to an embodiment, said cell lipids comprise triglycerides and/or phospholipids.

According to an embodiment, the method may further comprise purifying said bioproduct by separating co-extracted co-product lipids or a fraction thereof from the separated oil-soluble composition. According to an embodiment, said purifying comprises liquid-liquid extraction of the oil-soluble composition. According to an embodiment said liquid-liquid extraction uses a selective extractant. According to an embodiment said liquid-liquid extraction forms a purified bioproduct and a second extract, wherein said second extract is enriched in lipids, meaning that lipids/bioproduct ratio in said second extract is greater than that in the oil-soluble composition.

According to an embodiment, said selective extractant may comprise said solvent as a first solvent. According to an embodiment, said selective extractant may be a mixture of said first solvent with a second solvent. According to an embodiment, said second solvent may be more hydrophobic than said first solvent. According to an embodiment, said selective extractant comprises CO₂. According to an embodiment, said selective extractant comprises supercritical CO₂.

According to an embodiment, said purifying may form a purified bioproduct. According to an embodiment, said purified bioproduct comprises at least 10% astaxanthin, at least 15%, at least 20%, at least 25%, or at least 30%.

### EXAMPLES

### Example 1

A genetically modified astaxanthin-producing E. coli is cultured in a fermentation medium comprising glucose and a nitrogen source. A 110 OD (optical density) fermentation broth is formed. A sample of the broth is filtered to form separated cells and a cell-free solution. Both are analyzed. The astaxanthin content of the separated cells is found to be 8.7 milligram (mg) per gram dry cells. The cell-free solution's dissolved astaxanthin concentration is less than 20 ppm.

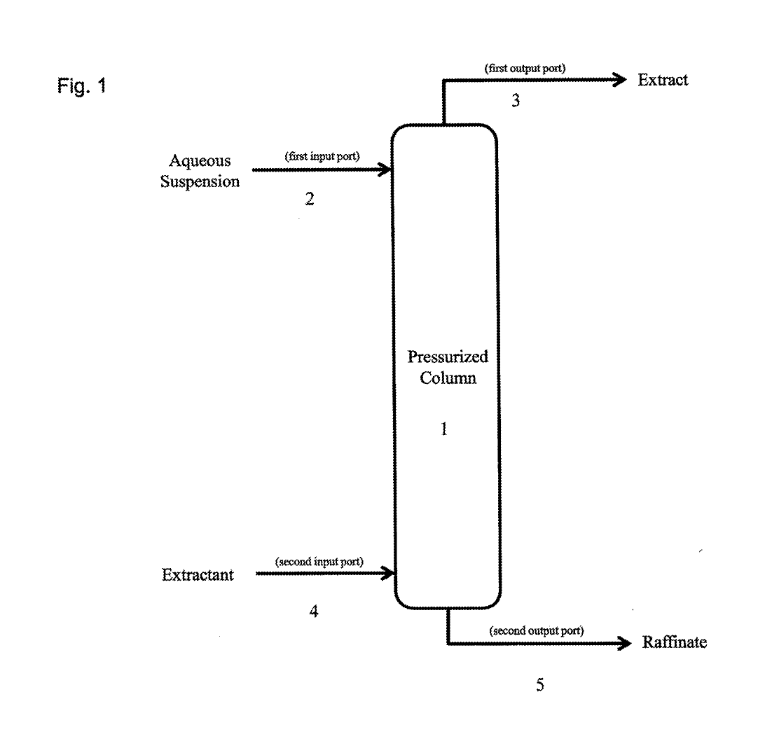

A packed column extraction column (1) is used for extraction. The column has a first input port (2) and a first output port (3) at its upper part and a second input port (4) and a second output port (5) at its bottom part, as schematically shown in FIG. 1. The column is configured to provide five theoretical extraction stages.

Butene is used as the extractant.

The fermentation broth is pumped into the column via the first input port at a rate of 50 milliliter/per minute (ml/min), while at 30° C. Based on the analysis, astaxanthin input to the column is 19 mg/min. The fermentation broth exits at the second output port at a rate of about 50 ml/min (due to minimal mutual miscibility of water and butene) and is collected as the raffinate. The extractant, pressurized to 5 bar, is pumped into the column via the second input port, at a rate of 30 ml/min, while at 30° C., and flows counter-currently to the incoming broth. It exits through the first output port and is collected in a pressurized vessel as the extract.

100 ml of the extract is opened to the atmosphere, whereby butene there evaporates. The rest of the extractant is removed by heating the residue to 50° C. The remaining residue is analyzed and found to contain 51 mg astaxanthin.

Accordingly, 81% of the astaxanthin in the broth is extracted.

### Example 2

The test in Example 1 is repeated with one modification. Extract input rate is doubled to 60 ml/min. This results in increasing the extraction yield to 86%.

### Example 3

The test in Example 2 is repeated except that the broth contains a genetically modified E. coli that produces omega-3 fatty acids. The omega-3 fatty acids content is 27 mg/g dry cells.

Extraction yield is 89%.

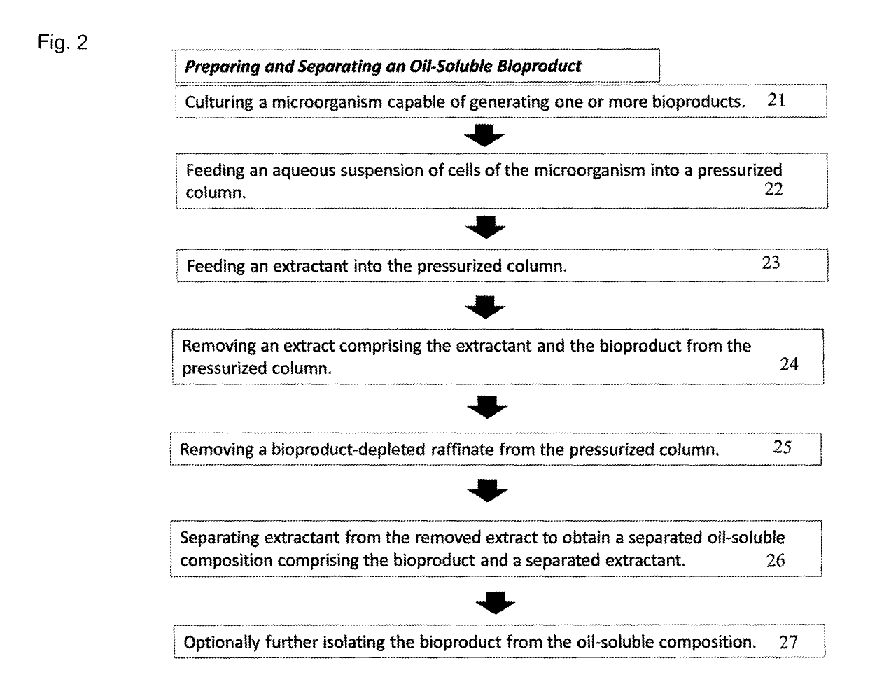

A flow diagram demonstrating an embodiment of the process is shown in FIG. 2. A microorganism is cultured which is capable of generating one or more bioproducts (21). An aqueous suspension of cells of the microorganism is fed into a pressured column (22). An extractant is fed into the pressurized column (23). An extract comprising the extractant and the bioproducts is removed from the pressurized column (24). A bioproduct depleted raffinate is removed from the pressurized column (25). The extractant is separated from the removed extract to obtain a separated oil soluble composition comprising the bioproduct and a separated extract (26). Optionally, the bioproduct is further isolated from the oil soluble composition (27).

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for producing an oil-soluble bioproduct composition, comprising
(i) culturing a host microorganism genetically modified for increased production of an oil-soluble bioproduct to form an aqueous suspension of cells comprising said bioproduct, which bioproduct is at least 30% intracellular;
(ii) providing an extractant comprising a solvent, which solvent has a boiling point at atmospheric pressure of less than 20° C.;
(iii) feeding said aqueous suspension into a pressurized column through a first input port;
(iv) feeding said extractant into said pressurized column through a second input port;
(v) removing an extract from said pressurized column through a first output port, wherein said removed extract comprises at least a fraction of said extractant and at least a fraction of said bioproduct;
(vi) removing a raffinate from said pressurized column through a second output port, wherein said removed raffinate is bioproduct-depleted; and
(vii) separating extractant from said removed extract to obtain a separated oil-soluble composition comprising the bioproduct and a separated extractant; wherein
a. the concentration of cells in the aqueous suspension fed to the pressurized column is in the range between 0.1 wt % and 10 wt % based on dry-cell weight;
b. the concentration of said bioproduct in said aqueous suspension fed to the pressurized column is in the range between 0.01 wt % and 1 wt %;
c. the solubility of said bioproduct in water at ambient pressure and 20° C. is less than 2 wt %;
d. the solvent forms at least 70 wt % of said provided extractant;
e. the solubility of said solvent in water at ambient pressure and 20° C. is less than 2 wt %;
f. said removed extract comprises water at less than 20 wt %; and
g. said removed extract is free of cells or comprises cells at a dry-cell weight that is less than 10 wt % of the dry-cell weight of the aqueous suspension.

2. A method according to claim 1, wherein said bioproduct is an isoprene derivative, fatty acid, fatty acid derivative, fatty alcohol, fatty alcohol derivative, glycolipid, and/or mixtures thereof.

3. A method according to claim 1, wherein said host microorganism is selected from the group consisting of bacteria, yeast, and microalgae.

4. A method according to claim 1, wherein said culturing comprises fermentation, wherein said fermentation forms a fermentation broth and wherein said aqueous suspension comprises said fermentation broth.

5. A method according to claim 1, further comprising rupturing cells in said aqueous suspension prior to feeding the aqueous suspension into said pressurized column.

6. A method according to claim 1, further comprising adjusting the cell concentration in said aqueous suspension prior to feeding the aqueous suspension into said pressurized column, which adjusting comprises at least one of water addition and water removal.

7. A method according to claim 1, wherein said solvent is CO₂, butene, butane, and/or propane.

8. A method according to claim 1, wherein the temperature of said column, the pressure of said column or both are within the super-critical range of said solvent.

9. A method according to claim 1, wherein said extractant and said aqueous suspension are fed at aqueous suspension/extractant weight/weight ratio in the range between 1/10 and 10/1.

10. A method according to claim 1, wherein said aqueous suspension is fed at a flow rate in the range between 100 L/h and 10,000 L/hr.

11. A method according to claim 1, wherein at least 80% of the bioproduct present in said aqueous suspension after culturing is recovered in the separated oil-soluble composition.

12. A method according to claim 1, further comprising purifying said bioproduct by separating co-extracted co-product lipids or a fraction thereof from the separated oil-soluble composition.

13. A method according to claim 12, wherein said purifying comprises extraction with a selective extractant.

14. A method according to claim 13, wherein said selective extractant comprises said solvent.

15. A method according to claim 13, wherein said selective extractant comprises CO₂.

16. The method according to claim 1, wherein providing an extractant comprises providing a recycled extractant previously utilized in a method for producing an oil-soluble bioproduct or an oil-soluble bioproduct composition.

17. A method according to claim 1, wherein said first output port is above said first input port.

18. A method according to claim 1, wherein said second output port is under said second input port.

19. A method for producing an oil-soluble bioproduct, comprising:
(i) culturing a host microorganism genetically modified for increased production of an oil-soluble bioproduct to form an aqueous suspension of cells comprising said bioproduct, which bioproduct is at least 30% intracellular;
(ii) providing an extractant comprising a solvent, which solvent has a boiling point at atmospheric pressure of less than 20° C.;
(iii) feeding said aqueous suspension into a pressurized column through a first input port;
(iv) feeding said extractant into said pressurized column through a second input port;
(v) removing an extract from said pressurized column through a first output port, wherein said removed extract comprises at least a fraction of said extractant and at least a fraction of said bioproduct;
(vi) removing a raffinate from said pressurized column through a second output port, wherein said removed raffinate is bioproduct-depleted;
(vii) separating extractant from said removed extract to obtain a separated oil-soluble composition comprising the bioproduct and a separated extractant; and
(viii) obtaining an oil-soluble bioproduct from the separated oil-soluble composition wherein
(a) the concentration of cells in the aqueous suspension fed to the pressurized column is in the range between 0.1 wt % and 10 wt % based on dry-cell weight;
(b) the concentration of said bioproduct in said aqueous suspension fed to the pressurized column is in the range between 0.01 wt % and 1 wt %;
(c) the solubility of said bioproduct in water at ambient pressure and 20° C. is less than 2 wt %;
(d) the solvent forms at least 70 wt % of said provided extractant;
(e) the solubility of said solvent in water at ambient pressure and 20° C. is less than 2 wt %;
(f) said removed extract comprises water at less than 20 wt %; and
(g) said removed extract is free of cells or comprises cells at a dry-cell weight that is less than 10 wt % of the dry-cell weight of the aqueous suspension
